(19) United States
(12) Patent Application Publication
(10) Pub. No.: US 2012/0291327 A1
(43) Pub. Date: Nov. 22, 2012

(54) BIOMETRIC QUICK RELEASE GUN LOCK

(76) Inventor: Michael Boutot, JR., Naples, FL (US)

(21) Appl. No.: 13/110,025

(22) Filed: May 18, 2011

(51) Int. Cl. F41A 17/00; A61B 5/117

(52) U.S. Cl. 42/70.11

(57) ABSTRACT

A quick biometric gun lock has a hinge located between two sides such that the two sides open and close about the hinge and a cavity between the two sides for reception of the finger protector and trigger mechanism of a gun such that the quick biometric gun lock encloses the trigger and finger protection area. A control shaft is attached to one of the sides having teeth for closure of the gunlock and extends into a hollow area of the other side. A control device in the other side is arranged to engage the teeth of the control shaft. This control device is either an electric motor or solenoid controlled by a microcontroller. For an electric motor the integral shaft moves a toothed servo shaft that is in contact with corresponding grooves of the electric motor's integral shaft; the solenoid has an integral shaft to control the gunlock.

### CROSS REFERENCE TO RELATED APPLICATIONS

N/A

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the United States Patent and Trademark Office patent file or records, but otherwise reserves all copyrights whatsoever.

### BACKGROUND

(1) Field of the Invention

Relating to improvements in the art of gun safety. More particularly, relating to improvement in gun locking devices.

(2) Description of the Related Art

US Patent Application Publication 2003/0141959 published on Jul. 31, 2003 describes a Fingerprint Biometric Lock; it was filed on Feb. 4, 2003 from a continuation-in-part of U.S. application Ser. No. 10/185,453, filed Jun. 28, 2002 which claims the benefit, of U.S. Provisional Application 60/302,154, filed Jun. 29, 2001. The aforementioned US Patent Application Publication 2003/0141959 published on Jul. 31, 2003 describes a Fingerprint Biometric Lock and is hereby INCORPORATED BY REFERENCE. The relevant portions of the cited patent are reproduced in the following paragraphs.

It is well known that rapid access to highly secured storage systems, such as gun safes and the like, is hampered when the system is secured using a keyed lock. It is also known that keys are easily misplaced, copied, and keyed locks are easily picked or drilled. Moreover, keyed locks do not limit access to a particular individual. A solution is to provide a fingerprint matching system that detects fingerprints and compares them with fingerprint data stored in a database for locking or unlocking safe storage systems.

Up to now a few such examples exist. For example, U.S. Pat. No. 5,579,909, to Deal, and U.S. Pat. No. 5,701,770, to Cook et al., disclose optical fingerprint readers combined with either a numeric keypad or fingerprint card backup access controls, respectively. Another example, U.S. Pat. No. 4,768,021, to Ferraro, discloses a pressure sensitive touch pad for access to a gun safe. A third such example, E.P. Pat. No. 0,976,897 A1, to Saito, discloses a lock and switch using a pressure-type fingerprint sensor. A fourth such example, U.S. Pat. No. 5,794,466, to Hungerford, et al., discloses a gun safe using a fingerprint access method with a mechanical override.

A controller card is provided to unlock the lock using a fingerprint scanning module. The controller implements all functions for enrolling, canceling enrolls and verification of valid fingerprints.

Referring now to FIG. 1, there is generally shown therein a diagram of the locking hardware according to the present invention. The hardware is controlled via a power management system, which enhances the durability of the fingerprint biometric lock when used with either fixture or portable storage applications. The lock is powered with a direct current voltage source 2, such as three internal dry cells, in a conventional direct current circuit. An external wall adapter 4 may, but need not, be provided for an alternating current power circuit in order to either prolong battery life, or for operation of the lock once the batteries have expired. An automatic transfer switch 6 on the input of both power supplies is then included to constantly monitor the direct 2 and alternating 4 current from each power supply. When alternating current is applied, the circuitry automatically isolates the batteries from the circuit until the alternating current supply is removed.

A +5V switcher 8 is used to provide +5V power to a fingerprint module 10 and an unlocking motor control unit 12. This supply is enabled or disabled by a central processing unit 16 which is programmed to control all functions on a control board. The output is preferably +5 Volts, at 500 mA, and a 2.7-5.0 volt input range.

A +3.3V switcher 14 is used to provide power to the microcontroller 16, a RS-232 level converter and a finger presence detection circuitry 18. The finger presence detector 20 is preferably an infrared detector which measures the transmission and reflection of infrared light, but may also be in the form of a micro-switch positioned near the fingerprint sensor 10. The output of the switcher 14 is specified at +3.3V, at 50 mA, and a 2.7-5 Volt input range.

The main central processing unit (“CPU”) 16 is preferably a Texas Instruments™ MSP430 family microcontroller. This device acts as a controller for all of the devices on the board. It monitors the circuitry, communicates with the fingerprint reader 10 and implements all enrollment processes for enablement of a fingerprint code. The CPU 16 controls the power going to all devices on the board. In a standby mode, the CPU 16 is in a sleep phase drawing minimal current for versatile use of the biometric lock in either fixture or portable applications. Approximately four times per second, the CPU 16 wakes up and powers up the finger presence detector 10 and then turns it off again. When a finger is detected, the CPU 16 powers up the +5 Volt switcher 8, the fingerprint sensor module 10 and the motor control unit 12. After the motor 24 has cycled, the CPU 16 shuts down the entire system and returns to sleep mode.

An enrollment button (not shown) is preferably positioned on the board and is used to enroll an enabled fingerprint for verification with an offered fingerprint. When the enrollment button is pushed, the CPU 16 powers up the +5 Volt switcher 18, the fingerprint module 10, and an RS-232 level converter to enroll an enabled fingerprint.

In a preferred embodiment of the locking system, the fingerprint presence detector 18 is an infrared reflective object sensor which detects the presence of a finger placed proximate to the fingerprint sensor 20. This device is mounted near the base of the fingerprint detector 18 in a “looking up” configuration. This device transmits infrared light for detection by its infrared receiver which is mounted next to the transmitter. Once a finger is placed close to the sensor 20, infrared light is reflected from the finger to the receiver resulting in a signal being sent back to the CPU 16. The CPU 16 then powers the fingerprint detector 22 at a rate of approximately four times per second.

The unlocking mechanism includes a motor driven cam shaft connected to the motor 24 with a cam aligned to pass over the controller board. The cam includes a small magnet mounted on it. A magnetic sensor is also mounted on the board in circumferential alignment with the magnet so that the magnet passes over the sensor upon rotation of the cam. When the magnet is aligned with the sensor, it sends a positive signal to the CPU 16 which indicates the exact angular rotation of the motor shaft.

A capacitive-based fingerprint sensor and module, such as the one marketed under the trademark Sagem Morphomodule, Sagem S.A., is preferred and requires RS-232 levels. A device which will generate these levels from the +3.3V power supply is positioned on the board to provide communication between the fingerprint module 10 and the CPU 16.

A buzzer or LED light may, but need not, be mounted on the board to provide the user with audible or visual prompts throughout the enrollment process, the enabled fingerprint removal process, and to provide a low battery indication.

The microcontroller 16 implements all functions on the board. The microcontroller 16 is used to perform fingerprint enrollment, fingerprint verification, enabled fingerprint removal, and power management. The program is stored in flash memory and is coded in the C programming language. Table 1 below lists and describes the inputs/outputs for the microcontroller 16.

TABLE 1

| Function | Input/Output | Description |
|---|---|---|
| Motor Excitation | O | Controls a Field Effect Transistor |
| | | That switches power to the motor. |
| Motor Position | I | A hall effect sensor gives an |
| | | active signal when the |
| | | motor completes on revolution. |
| +5 V Enable | O | Controls a FET which turns |
| | | on the 5 V supply |
| Fingerprint | O | Controls a FET that switches power |
| Sensor Enable | | to the fingerprint sensor. |
| Finger Detect | O | Controls a FET that switches power |
| Enable | | to the finger detect circuitry. |
| Buzzer | O | Drives the buzzer for user prompts |
| Wall Power | I | Feedback from the 5 V switcher |
| | | indicating that the voltage has |
| | | reached full value. |
| RX | I | Serial communication from the |
| | | fingerprint reader. |
| TX | O | Serial communication to the |
| | | fingerprint reader. |
| Vbatt | I (Analog) | Port which monitors the battery |
| | | voltage for low battery. |
| Pushbutton | I | Pushbutton used to start the |
| | | enrollment sequences. |

FIG. 2 illustrates the program logic flow for a single sample fingerprint enrollment. Here, the user first presses 30 the push-button on the board. This action wakes up 32 the CPU, turns on the fingerprint reader 34 and sounds the beeper for a single beep 36. The CPU enables power to the fingerprint reader and engages 38 it into an enroll mode 40. After five seconds, the CPU preferably turns off 42 the +5V supply and fingerprint reader, and beeps 44 the buzzer three times to indicate a valid fingerprint read, or five times 50 to indicate an invalid fingerprint. Another two beeps 48 are indicated, where a valid condition has been evaluated 40, to indicate to the user that the now enabled fingerprint has been successfully stored in a flash memory 46. The user may now unlock the lock by placing their offered fingerprint on the fingerprint sensor. Where the evaluation step 40 results in a bad enroll, the CPU sounds 50 the buzzer 5 times and turns off 52 the +5V power supply and fingerprint reader.

FIGS. 3, and 4 show an alternative program logic flow for an even more secure enrollment method of an enabled fingerprint. Here, in a three sample enrollment method, the user presses 60 the push-button on the board which wakes up the CPU, turns on the +5 V power supply 62, turns on 64 the fingerprint reader, and operates the buzzer 66 for a single beep (FIG. 4). The CPU enables power to the fingerprint reader and, as described above, puts it into an enroll mode 68, signaled with a single beep. Within five seconds, the CPU will beep the buzzer 70 a single time for a valid fingerprint or five times 72 for an invalid fingerprint. For a valid fingerprint, the controller waits for the user to remove their finger 74. The user must then place their finger 76 on the sensor a second time and repeat the above sequence. Once three successful samples have been taken 78 the CPU will sound 80 the signal three beeps. The CPU does not accept three invalid fingerprints 78, 82, throughout this sequence, before signaling 84 a beep six times to indicate that the user must press the push-button and start the sequence over. For the valid condition, the enabled fingerprint data are written into the flash memory 86, another two beeps are signaled 88 after the new fingerprint has been successfully stored in the flash memory, and the +5V power supply and fingerprint reader are turned off 90 (FIG. 3). The user may now open the safe by placing their offered fingerprint on the finger print sensor.

Referring now to FIG. 5, where it is shown the program logic flow of the fingerprint verification method, the finger presence detection circuitry is preferably turned on 92 approximately four times a second 94 for detecting 100 the presence of an offered finger. Once an offered finger is detected, the CPU powers up 102 the fingerprint sensor 104 and enables it 106 into a verify mode 108. When the offered finger print data match an enabled finger print data the fingerprint reader is powered off 110, and the CPU, which enables power 112 to the motor control unit, drives the motor until a Hall Effects sensor indicates 114 that the motor has completed its rotational travel. When an invalid fingerprint is indicated, the CPU signals the reader to turn off 116 the fingerprint reader and the +5V power supply ending the verification method.

FIG. 6 shows the program logic for removal of an enrolled fingerprint from the flash memory. To remove or erase all enrolled fingerprints from the fingerprint reader the user must press 118 the push button and hold 120 it for five seconds. If so, the CPU turns on the +5V power supply 122, turns on the fingerprint reader 124, and sends 126 a delete command to the fingerprint reader. When all enrolled fingerprint data have been erased 128 from the flash memory, the CPU turns off 130 the +5V power supply and the fingerprint reader, and sounds six times 132 to indicate deletion of the data.

FIG. 7 shows the program logic for power management system. The CPU controls all power management functions on the board and begins in a sleep mode 140.

It then determines whether wall power is applied 142, and, if so, disables the +5V power supply 144. If wall power is not applied, it enables the +5V power supply 146, wakes up every fifteen minutes 148 and measures 150 the battery voltage, and determines 152 whether the battery voltage has fallen below 2.7 Volts. If the battery voltage is less than 2.7 Volts the CPU goes back into the sleep mode, and sounds 154 the beeper once every fifteen minutes.

FIG. 8 is a schematic diagram of the personality board the motor control circuit. In the preferred embodiment, the controller board 160 is preferably mounted directly to the motor 162, but may also be used to control a stepper motor, solenoid or magnetic release. A Hall Effects sensor is mounted in a position that can monitor the magnet attached to the cam on the +3V DC motor. The enrollment push button 164 and buzzer 166 are also mounted directly to the board 160. The finger print presence detector (not shown), alternating current adapter connector 170, 6V DC power supply 172, and fingerprint reader 168 are all connected through the board via a wiring harness in any manner well known in the art.

### EXAMPLE

The following example describes use of the fingerprint biometric lock for use in unlocking a portable gun safe.

Referring now to FIGS. 9 and 10, wherein like numerals represent like features there is shown generally therein a high security portable gun safe unit 200 for use with the biometric lock as its single means of entry. The gun safe 200 includes a top wall 202 having a hinged door 204, a bottom wall 206, side walls 208, a front wall 210 and a back wall 212. The top wall 202 is hinged with torsion springs 214 for spring loaded opening of the hinged door 204 when unlocked. The bottom wall 206 includes a mounting plate 216 secured with mounting screws 218 for secured attachment of the safe 200 to immobile objects when used in fixture applications. Also included is the above described electronic circuit that restricts the unlocking of the locking mechanism. The locking mechanism includes a latch opener 220 biasing against one or more sliding bolts 222 and compression springs 224 driven by a motor 226 driven cam 228 that secure the hinged door 204 in a locked position (as shown). The motor control unit includes the motor driven cam shaft with the cam 228 aligned to pass over the controller board 230. The cam 228 includes small magnet 232 mounted on it. A magnetic sensor is mounted on the board 230 in a position adjacent to the magnet 232 such that as the magnet passes over the sensor it sends a positive signal to the CPU for indication of the exact angle of the shaft.

In the preferred embodiment, the fingerprint sensor is a capacitive-based fingerprint sensor 234, and is wired through a slit opening in the exterior of the safe's top wall 202 for detection of an offered fingerprint. The fingerprint sensor 234 is connected to a scanning module 236 connected directly below the sensor to the interior of the safe. In combination, the sensor 234 and module 236 are used to detect and process the presence of a fingerprint. This fingerprint scanning module 236 provides for enrollment of one or more digital fingerprint template data for entry into the flash memory of the fingerprint scanning module, as an enabled user. The module 236 is also used for verification of enrolled users and signals the electronic movement restricting circuit upon verification of an enrolled user. Three D-Cells 238 are contained in a conventional battery holder 240 and are used to provide a direct current to the power management circuit which, in turn, provides power to the locking mechanism and the fingerprint reader.

The safe is preferably constructed of 18 gauge steel on each side, top, bottom and door but may be constructed of any tamper resistant material, such as any high impact or ballistic material which are well known in the art. The top wall 202 steel has been formed such that the door 204 is offset to the inside by one-half inch to discourage prying of the door 204. The door 204 is spring loaded for immediate access to the interior of the safe 200 upon opening of the unlocking mechanism. The position of the hinge can be moved depending upon the desired application. A double bolt system 222 is used in latching the door 204 in a closed position and is controlled by a servo motor 226. A slot extends top middle of the top wall for the wires from the fingerprint reader to connect to a relay board located on the inside top middle of the door.

The locking mechanism is located on the inside of the door. This mechanism includes the servo motor 226 that moves the two locking bolts 222 in and out of aligned recesses in the hinged panel 204. The motor 226 is activated by a relay movement restriction circuit attached to the fingerprint reader board 236. When a finger is placed on the reader 234 and verified as an enrolled user, the board sends a signal to the relay which in turn allows electricity to be sent to the motor 226. This electric signal will drive the motor 226 to rotate the cam 228 away from the bolts 222 whereby the springs 224, normally compressed when the door is latched, expand and move the bolts 222 outwardly from the latch recesses for unlocking the door.

In operation, one uses the gun safe to secure items in a highly secure but rapidly accessible manner. Mounted on the inside of the safe is the enrollment button 242 which operates, as described above, in either a one or three step enrollment sequence, for enrolling a fingerprint data into the flash memory of the fingerprint module. The whole fingerprint is not stored, but an algorithm is used to determine minutiae points from the fingerprint points, of the fingerprint, and stores them as a template in the flash memory. Once the template has been stored, the enabled fingerprint is now the exclusive means of gaining entry to the safe. In this mode, the enrollee simply places their offered finger on the fingerprint reader and, once it has been verified as an enabled fingerprint, the verification unit sends a signal to the relay. The relay then allows electricity to flow to the motor that drives the locking bolts outwardly.

The Keogh et al Patent Application Publication 2003/0141959 published on Jul. 31, 2003 provides a biometric safe having flash memory, power supply, CPU and the many other components described above in paragraphs 4-30. The Keogh application publication teachings are directed to storage locker type safes where the entire item is stored therein. Its size is cumbersome and takes up a great deal of space. Thus, what is needed are novelties that can secure a firearm, reduce the size of the protection device to a more practical level and that can still provide the biometric functionality found in the Keogh et al. application publication.

More particularly, gun safety is the number one need for a gunlock but several problems immediately arise in that the gun must be locked properly and it must also have the ability to be unlocked rapidly without excessive delay. If there was a great deal of delay, a gun owner might find himself or herself without sufficient time to unlock the gun. As a result, family members or patrons might suffer harm as the result of an intruder entering his or her home or business. Another problem that impacts the easy quick unlocking of a gun is the size of the lock. If the lock is large and cumbersome totally enclosing or greatly enclosing the weapon then a gun owner wastes time fumbling with the lock in an emergency situation. This is not the way to rapidly respond to threats. Worse than that is that he or she must run to the location of the locker or safe wasting valuable time in an emergency situation. Thus, what is needed is a mechanism that enables a gun owner to quickly respond to threats and that makes the gun readily available and easily portable from one location to another and that can secure the lock from unauthorized users.

### BRIEF SUMMARY OF THE INVENTION

A quick biometric gun lock comprising a hinge located between two sides such that the two sides open and close about the hinge and a cavity between the two sides for reception of the finger protector and trigger mechanism of a gun wherein the quick biometric gun lock encloses the trigger and finger protection area. A control shaft attached to one of the sides having teeth for closure of the gun lock. A control device in the other side for engaging the teeth of the control shaft. The quick biometric gun lock of claim 3 wherein the control device is an electric motor and integral shaft moving a toothed servo shaft that is in contact with corresponding grooves of the electric motor. A stopping disk integral with the servo shaft and a stop integral with the interior of the gun lock. Further, the quick biometric gun lock claim wherein the control device is a solenoid and integral shaft. A stopping disk integral with the integral shaft and a stop integral with the interior of the gun lock. A concave portion extending from one side of the gun lock having a biometric fingerprint sensor. Also included is a low battery detection speaker and a low battery detection light. Other components include a USB port utilized for both data transceiver operations and for recharging facilities; a conventional power supply recharging port; a rubber seal affixed to the gun lock's external surface. Other major components include a spring mounted bumper connected to one of the sides and operating against the two sides of the gun lock so as to open the gun lock; a spring mounted internally to the two sides and configured to operate against the internal surface of the exterior face of the sides. The last major component is one or more protrusions integral with an interior external surface of one side and matching cavity(-ies) on the other side for reception of the protrusion(s).

A quick biometric gun lock device comprising two half portions forming a main body of the gun lock connected by a hinge and integral with a portion extending from one side of the gun lock having a biometric fingerprint sensor. An oblong spring mounted bumper connected to one half portion. An internal spring connected to the hinge and in contact with both half portions.

A quick biometric gun lock device comprising two half portions forming a main body connected by a hinge and a device for holding the two portions together in cooperation with a toothed shaft integral with one portion and extending from one portion to another.

### BRIEF DESCRIPTION OF THE SEVERAL VIEWS OF THE DRAWINGS

FIGS. 1-10 describe the Prior Art US Patent Application Publication 2003/0141959 published on Jul. 31, 2003 for a Fingerprint Biometric Lock of Keogh et al.

FIG. 1 is a PRIOR ART diagram showing the power management to the biometric locking system for use in either in either fixture or portable highly secure applications.

FIG. 2 is a PRIOR ART flow chart showing the enrollment steps for enabling a valid fingerprint for access to the biometric locking system.

FIG. 3 is a PRIOR ART flow chart showing an embodiment for single step enrollment for enabling a valid fingerprint for access to the biometric locking system.

FIG. 4 is a PRIOR ART flow chart showing an alternative embodiment of a three step enrollment for enabling a valid fingerprint for access to the biometric locking system.

FIG. 5 is a PRIOR ART flow chart showing the preferred steps for verifying an enabled fingerprint and powering the unlocking mechanism for access to the object to be secured.

FIG. 6 is a PRIOR ART flow chart showing the preferred steps for deleting an enabled fingerprint for removing access to the object to be secured.

FIG. 7 is a PRIOR ART flow chart showing the preferred steps for extending battery life when wall and battery power are applied.

FIG. 8 is a PRIOR ART diagram of the control board according to the preferred embodiment of the cited art.

FIG. 9 is a PRIOR ART schematic bottom view drawing of a highly secure portable gun safe which is accessed using the fingerprint biometric lock according to the cited art.

FIG. 10 is a PRIOR ART side view of the gun safe shown in FIG. 9.

FIG. 11 illustrates a firearm having the Biometric Quick Release Gun Lock arranged about the trigger mechanism region of the preferred embodiment.

FIG. 12 illustrates the Biometric Quick Release Gun Lock from a front view showing various items associated with the preferred embodiment.

FIG. 13A-B illustrates the Biometric Quick Release Gun Lock from back views showing various items associated with the preferred embodiment. FIGS. 13A-B shows the fingerprint reader located on different sides of the Lock so as to show the different ways the invention can be laid out. FIG. 13 C shows a top view of the Gun Lock.

FIG. 14 illustrates a motor and gear utilized in the preferred embodiment that are internal to the Biometric Quick Release Gun Lock.

FIG. 15 illustrates a solenoid utilized in an optional version of the preferred embodiment that are internal to the Biometric Quick Release Gun Lock.

FIG. 16a illustrates two halves of the gun lock 1600 and 1610 that have a spring 1620 associated there between. FIG. 16b illustrates a cutaway showing the interior of the gun lock. FIG. 16C shows the positioning of protrusions and matching cavities; the tongue protrusions fit into matching cavities thereby ensuring that the device will not disengage from the trigger guard.

### DETAILED DESCRIPTION OF THE INVENTION

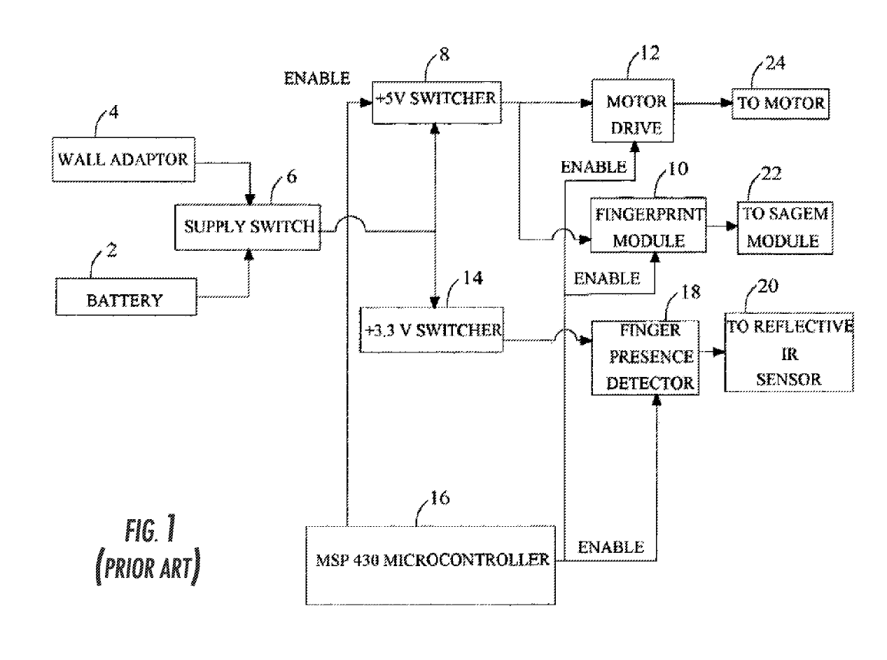

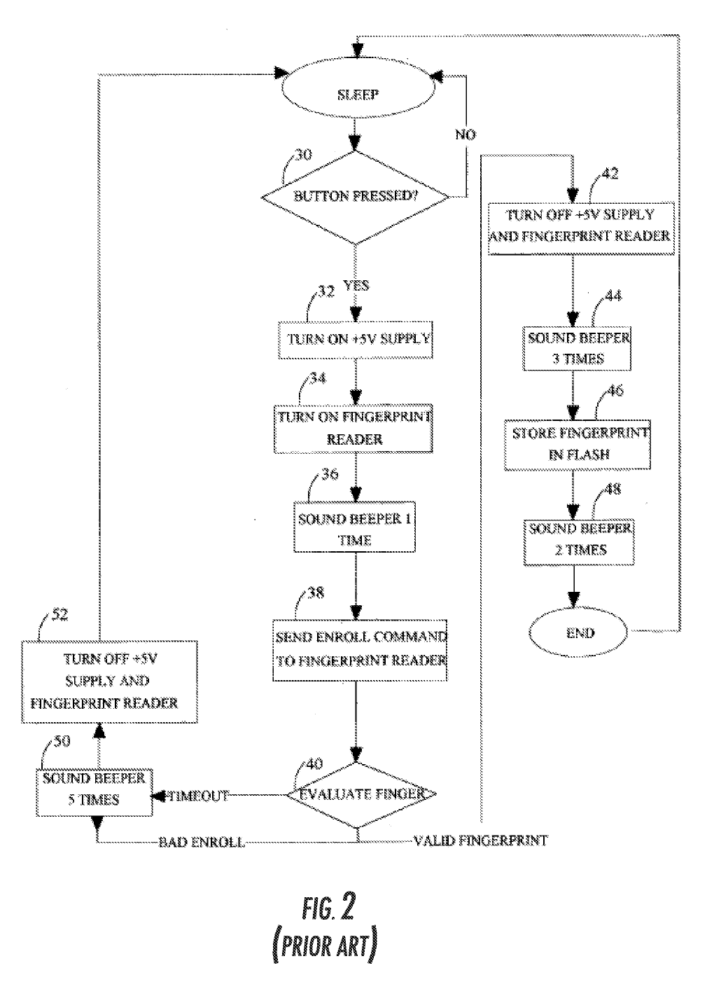

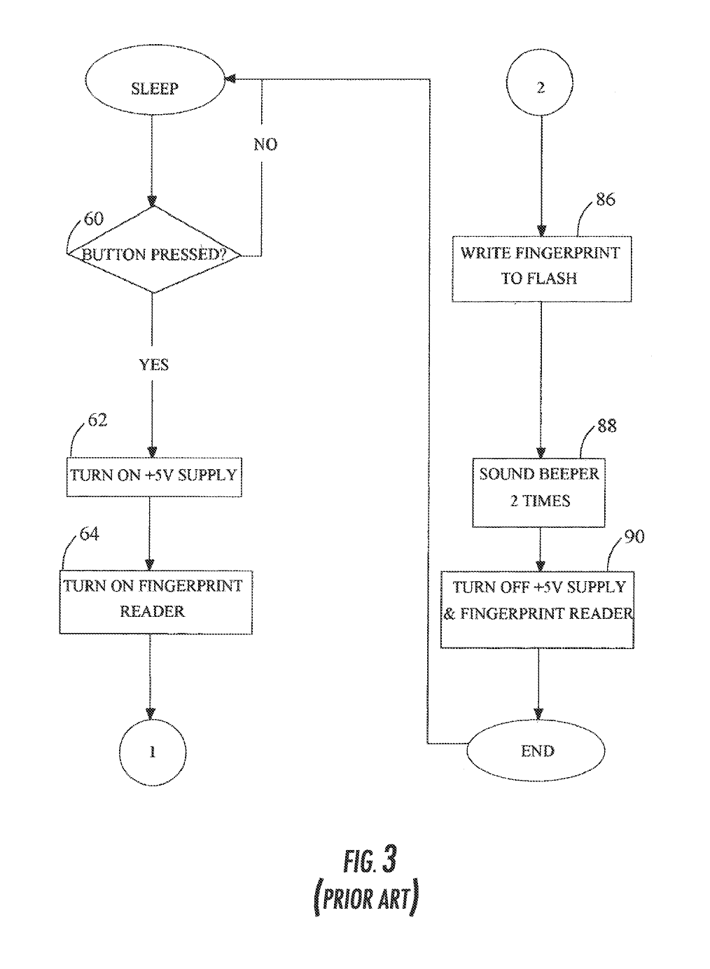

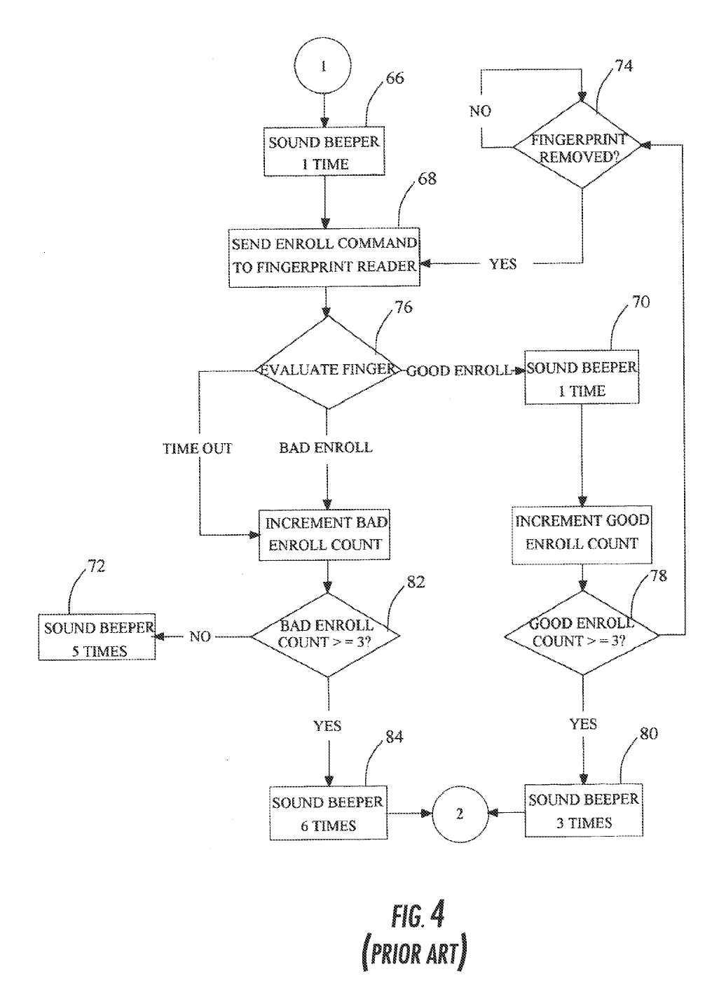

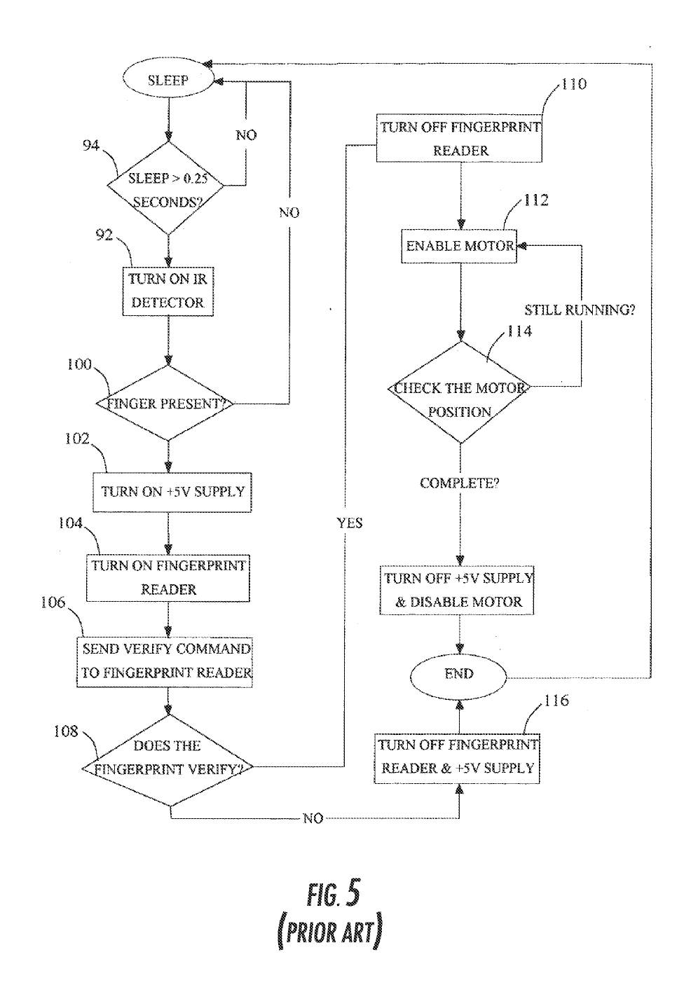

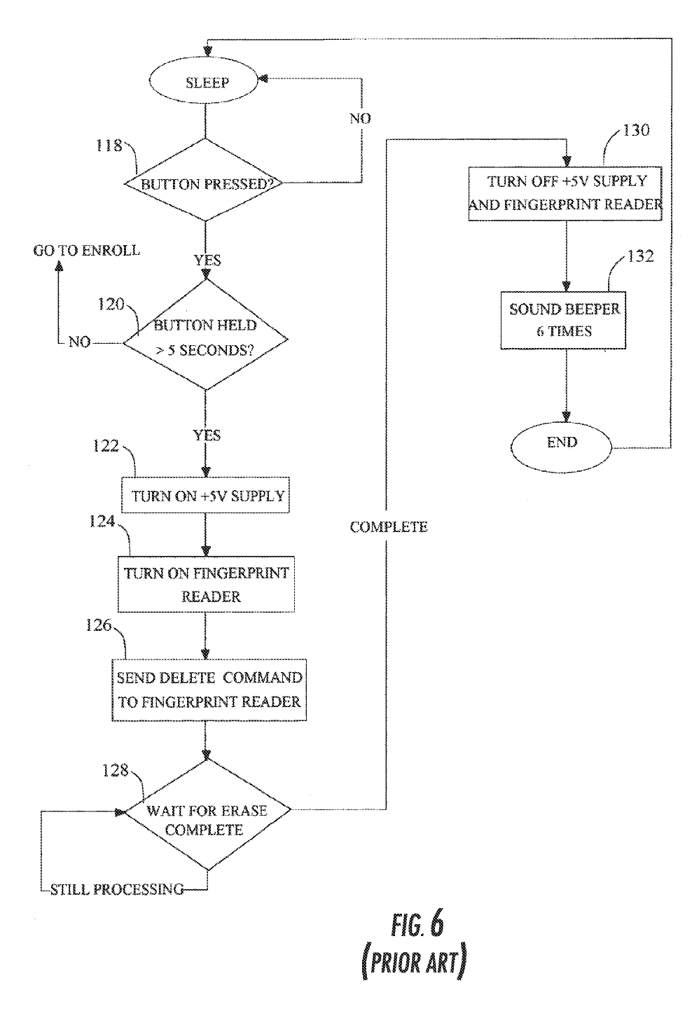

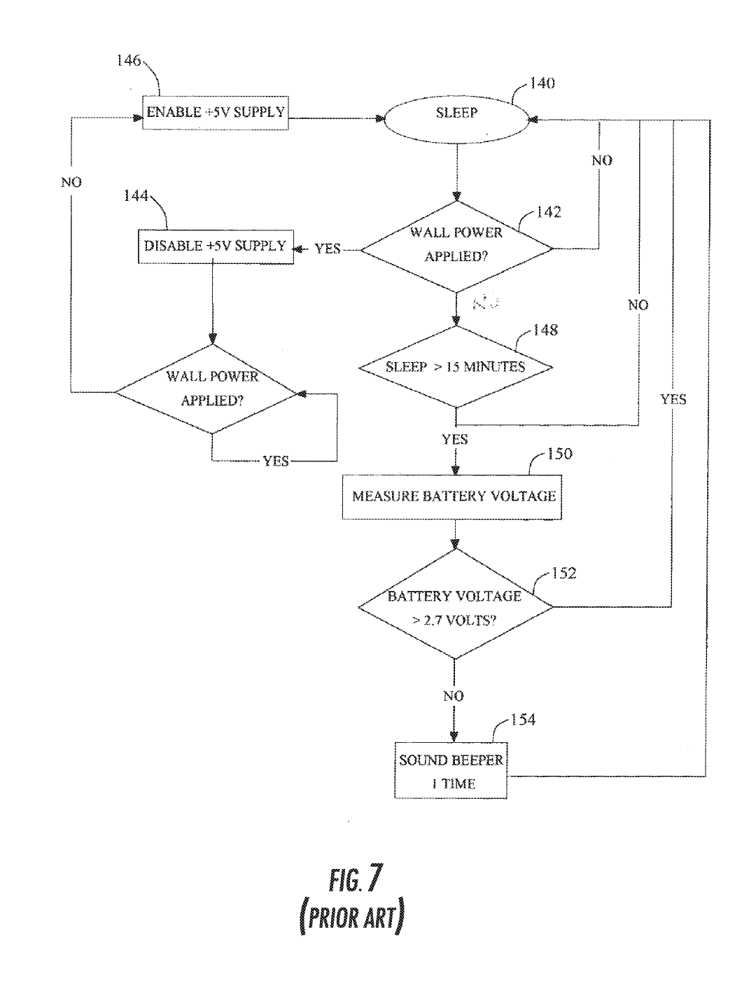

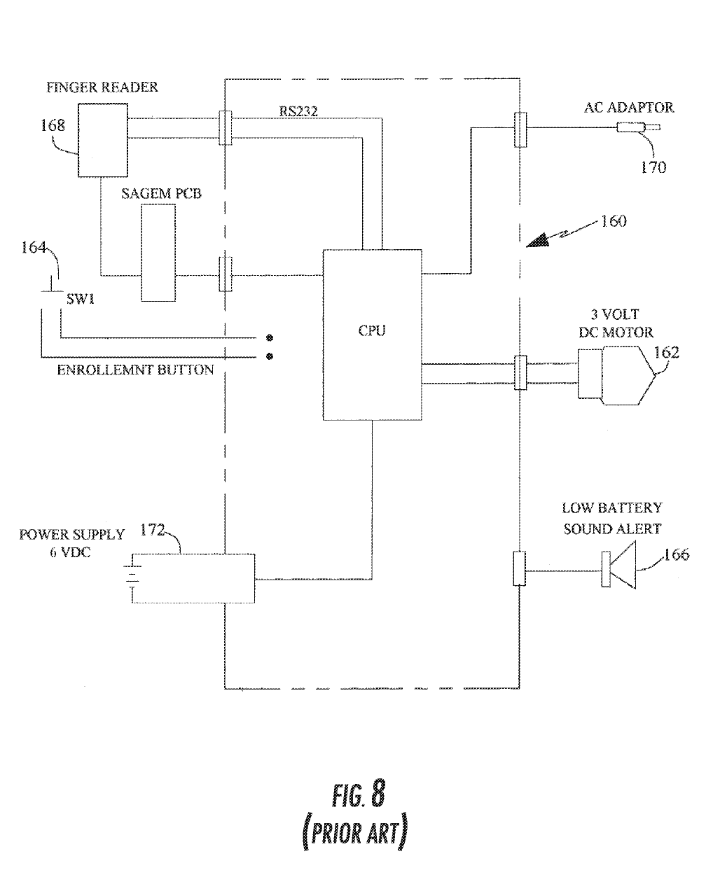

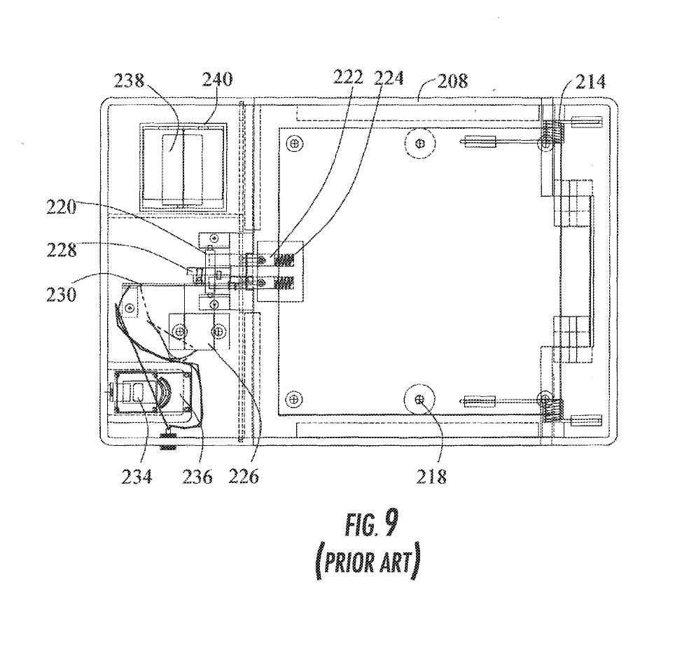

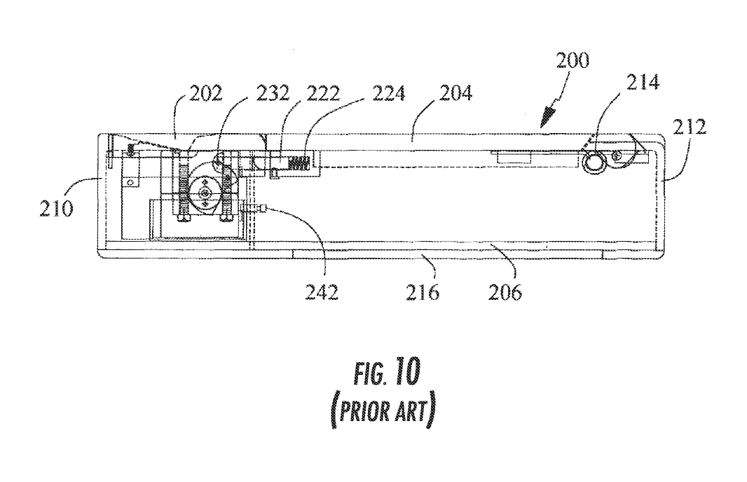

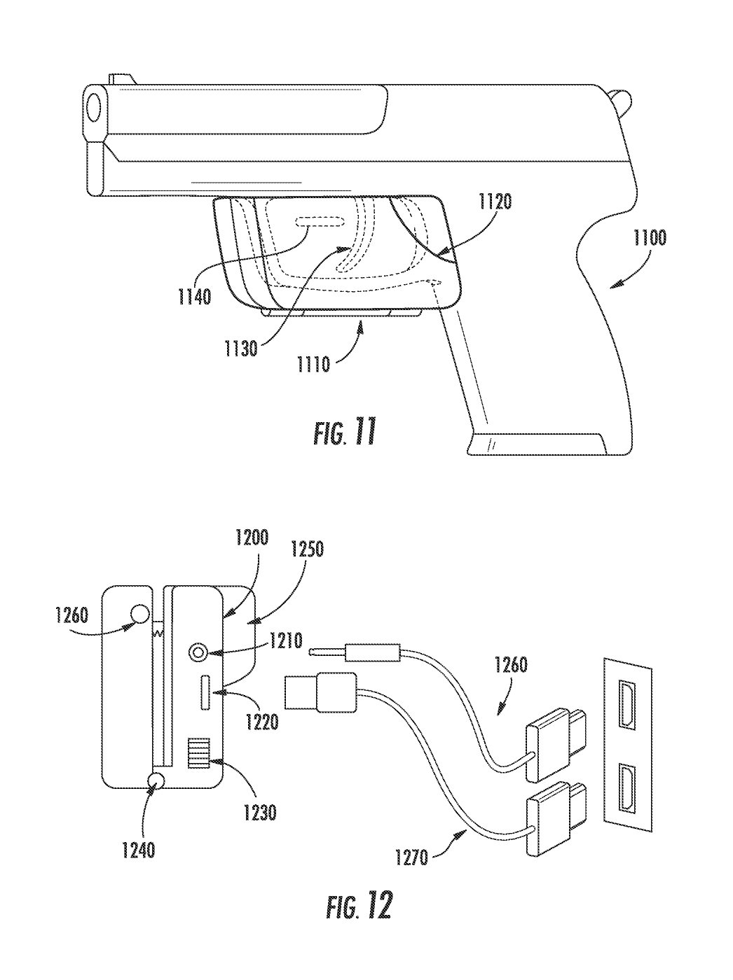

FIGS. 1-10 describe the prior art U.S. Patent Application Publication 2003/0141959 published on Jul. 31, 2003 for a Fingerprint Biometric Lock of Keogh et al. The electronic, computer hardware, power supply, flash memory, biometric detection and other components of the aforementioned patent application are hereby INCORPORATED BY REFERENCE. The principle electrical, electronic, computer hardware and software of Keogh et al. are to be utilized in the instant invention BIOMETRIC QUICK RELEASE GUN LOCK. Thus, the preferred embodiment described hereinafter primarily relates to the mechanical hardware that are novel in construction and use; additionally, the novel combination of the herein described mechanical hardware with the aforementioned electrical, electronic, computer hardware and software of the Keogh patent application publication are also included in the invention.

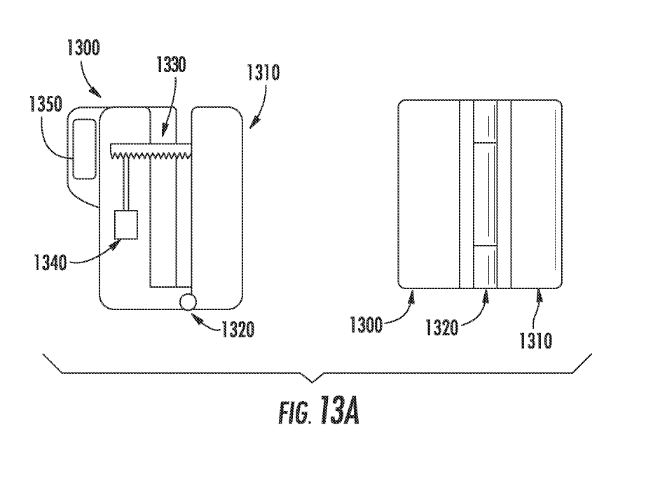

FIG. 11 illustrates a firearm 1100 having the Biometric Quick Release Gun Lock 1110 arranged about the trigger mechanism 1130 region of the preferred embodiment. The lock has two enclosed hollow halves or shells that encapsulate the trigger mechanism and finger protector between them as shown in the figure; each hollow half is formed somewhat greater than the size of the trigger guard and trigger and are arranged to abut the front side of a gun handle as shown; further, each half has perforations and internal mounts that are described more fully with regards to the following description. Additionally there is a thumbprint scanning biometric area 1120 shown as a concave region integrated on one half side of the Lock 1110. The thumbprint scanning biometric area 1120 is optionally arranged on the other side as a complementary image of the way it is represent in the figure so as to provide the same service for the left handed individuals. The two halves of the Lock 1110 are connected hinge style at the bottom as shown in the following figures.

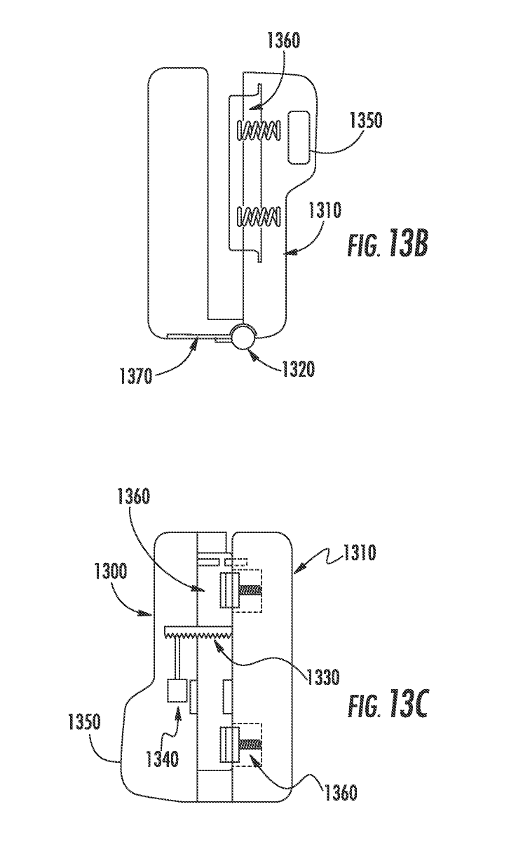

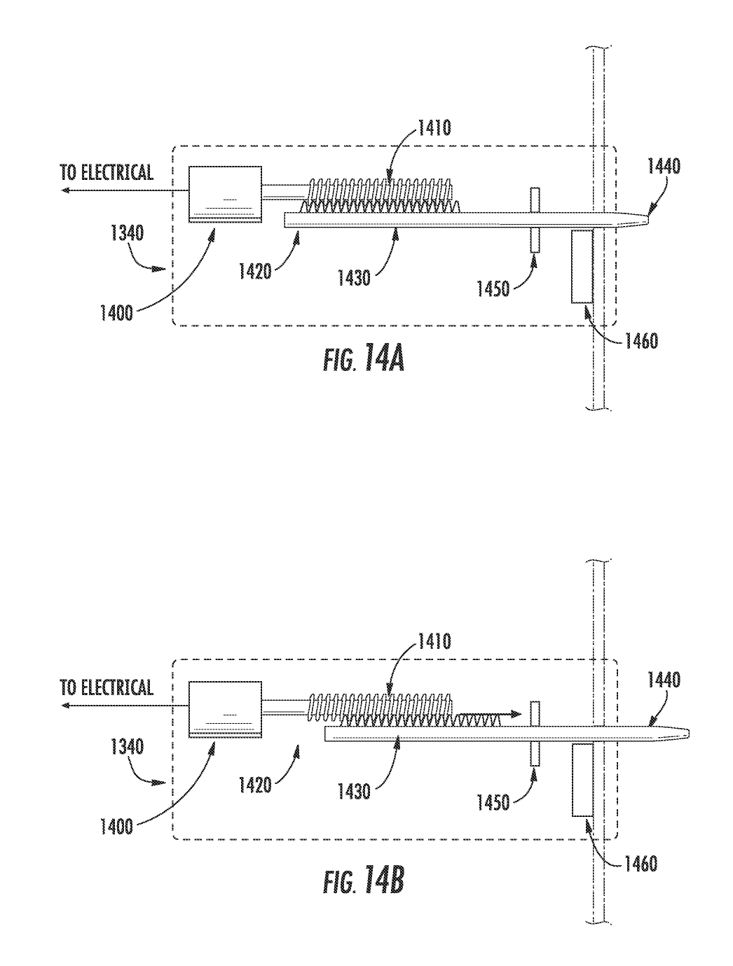

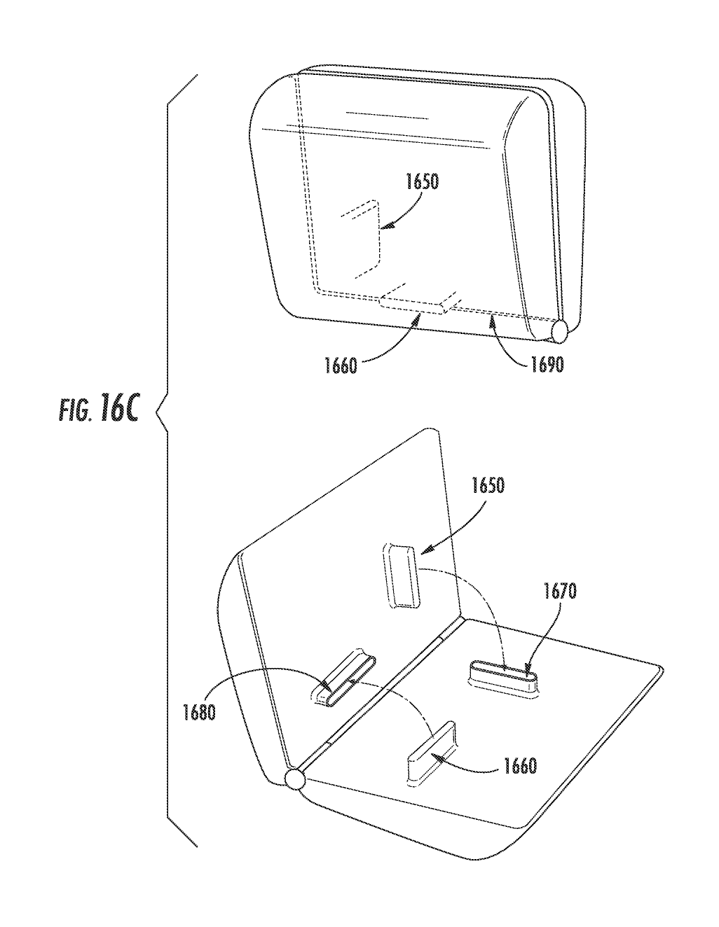

FIG. 12 illustrates the Biometric Quick Release Gun Lock 1200 from a front view showing various items associated with the preferred embodiment. As previously described, the two halves of the Lock 1200 are connected hinge style at the bottom as shown by a welded or screwed on hinge 1240. Near the hinge location is a spring (FIG. 13B, 1370, FIG. 16A, —threaded internally between the two halves, wound about the hinge and across an opening in the hinge cylinder portions and positioned internally against the internal surface of the most outward faces on either side of the gun lock halves so as to assist the springing motion to open the two halves) that permits the rapid opening of the Biometric Quick Release Gun Lock 1200 when a properly authorized thumbprint has been presented to the concave thumbprint scanning biometric area 1250; as shown in the figure, this concave area 1250 presents a mechanism through which a user may gain access to the gun deactivating the electro-mechanical mechanism that seals the lock and permitting the spring discussed above to press against the two halves opening the lock that is easily stored away. Thus, a gun owner can gain access to the gun easily in an emergency situation. A user also uses the concave thumbprint scanning biometric area 1250 to store thumbprints as describe above in Keogh. The entire lock is made of lightweight material such as aluminum or lightweight steel alloy and has a protective shock absorbing coating on its exterior (shown in another drawing). The electronics of the device are run off of internal batteries 1230 inside the lock. Additionally, a light 1260 is provided externally that indicates to a user a low battery condition and it is connected across the same circuitry that a complementary low battery sound indicator or speaker not shown in the drawing but connected appropriately as shown above with regards to Keogh. A user-friendly USB port 1220 comes with a associated wire and transformer 1270 or standard power port 1210 having a wire and transformer 1260 are provided as shown for recharging of the batteries 1230. The USB port can also be connected to a computer USB port for the recharging of the internal batteries or for data downloads directly to a personal computer or portable computer. A microcontroller (not shown) or cpu inside of the lock 1200 controls the operation of all software, biometric scanning, registering and analysis of the fingerprints and storage on a flash memory or similar memory as shown in Keogh.

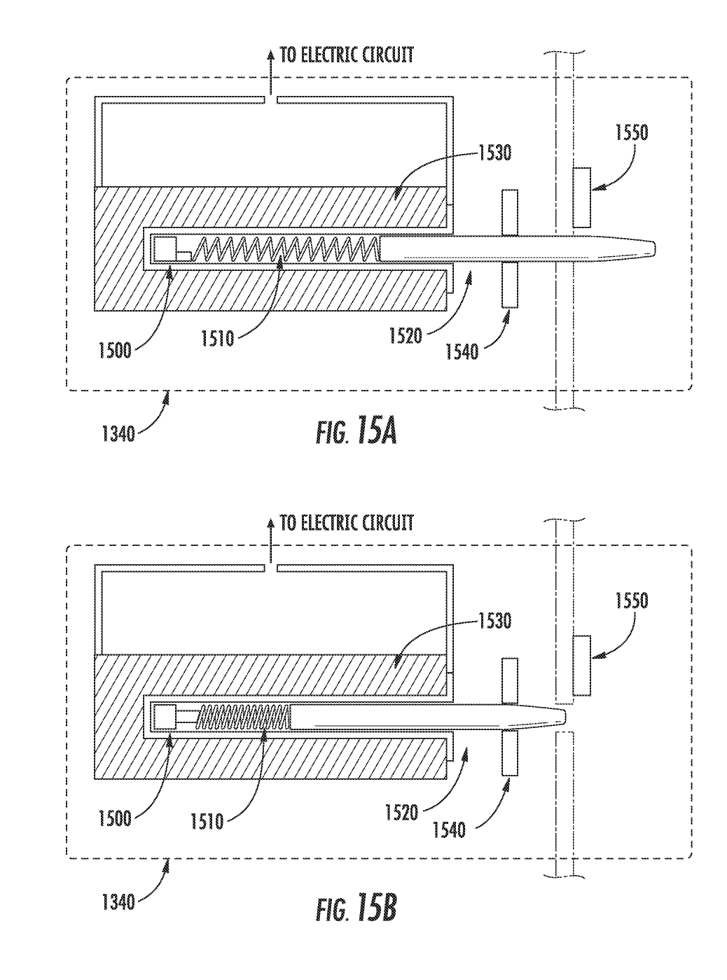

The halves of the gun lock 1200 are hollow and have convenient mounts that are integral with the halves or themselves welded to the interior surface of the device inside of each half for circuit board, battery holders, microcontroller, shafts and servo shaft, and other components; in lieu of a direct mount, the various devices are welded to the internal surface or are welded using shaped mounting devices that are form fitted to each device as an example in FIG. 16B 1340. The shafts, servo shaft etcetera have as many shaped mounting devices as necessary to restrict the motion of the devices forward and backwards according to the intended use and shaped guide mounts that hold the shafts in a controlled fashion so as to manage their operational sliding; this includes mounts with perforations on one side of the mount for allowing contact between the motorized shaft and the toothed shaft as in FIG. 14a. Additionally, the battery mount can optionally have an internal access door as in a cell phone or PDA or similar device that is located at a face between the two halves and adjacent to the trigger mechanism so as to restrict access unless the lock is disarmed.

FIG. 13A illustrates the Biometric Quick Release Gun Lock from a back view cross-section showing various items associated with the preferred embodiment. The side of the device form two halves as described herein and form interior surfaces for the welding of the various components and or with integral raised weld points and or welded mounting devices. When solely using the plain interior surface the welds can be directly to a component or assisted with a form fitted guide mount as shown in FIG. 16B, 1340. The Biometric Quick Release Gun Lock is shown having two halves 1300 and 1310 arranged as one item about an internal hinge 1320. A shaft 1330 having teeth extends out from the right side to the left side in the drawing. The shaft is integral with the rest of the external shell of the gun lock or welded onto it. It is made of materials identical or optionally similar to the materials that the gun lock itself is made from; typically an aluminum or lightweight steel is used. A control device 1340 regulates the position of the shaft 1330 as it enters a hollowed area. When the regulation device 1340 impact the fissures in each tooth it holds the Gun Lock closed in place thereby prohibiting access to the gun. The shaft 1330 is arranged to sit in front of or behind the trigger mechanism as shown in FIG. 1 thereby preventing a user from accessing and the firing of the weapon. The toothed shaft 1330 proceeds from the half 1310 and enters the half 1300 through a hollowed out portion that allows it access to the inner workings of the half 1300 where the device 1340 waits to engage and disengage it.

FIG. 13B illustrates the fingerprint reader 1350 located on a different side of the Lock so as to show the different ways the invention can be laid out. One of a pair of external spring mounted bumpers 1360 are shown that help deploy the device into opening the gun lock; once an acceptable fingerprint has been brought into contact with the fingerprint reader the locking mechanism 1340 disengages from the toothed shaft 1330 thereby opening the gun lock. The springs are welded onto the oblong bumpers 1360 and welded on the opposite end into the external face of the inner side of the half 1310. Of course, they could be located on the side 1300 solely or located on both sides 1300 or 1310. An internal spring 1370 is located near the hinge location. This spring shown in FIG. 13B as item 1370 and in FIG. 16A as item 1370 is threaded internally between the two halves, wound about the hinge or internal to the hinge and across an opening in the hinge cylinder portions and positioned internally against the internal surface of the external faces on either side of the gun lock halves. This spring passes through appropriate holes in each half 1300 and 1310 as well as being wound internally or externally of the hinge with appropriate exit and entry holes as necessary in the particular implementation. This helps the springing motion to open the two halves thus permitting the rapid opening of the Biometric Quick Release Gun Lock 1200 when a properly authorized thumbprint has been presented to the concave thumbprint scanning biometric area 1250. FIG. 13 C shows a top view of the Gun Lock. Item 1360 depicts the spring mounted bumpers from a top view also illustrating the space between the two halves within which the trigger guard and trigger slide into. Item 1350 shows a different location for the biometric scanning area than that shown in FIG. 13B. Additionally, a different orientation is shown for the motor or solenoid controlled device 1340. As one can appreciate the orientation of the motor or solenoid requires an appropriate rearrangement of the teeth on the integral shaft. Indeed the shaft itself 1330 is located typically in front of the trigger when the Lock is closed or optionally behind it; another option has the shaft located outside of the trigger guard area itself.

FIG. 14a illustrates a motor and gear utilized in the preferred embodiment that are internal to the Biometric Quick Release Gun Lock. The control device 1340 of FIG. 13 is shown in FIG. 14 as a electric motor 1400 integrated with a rotating worm type shaft or helical shaft 1410 having a continuous groove about the integral shaft as shown. This shaft 1410 actuates the motion of a corresponding servo shaft made up of several unique portions 1420, 1430 and 1440. The motor 1400 is bolted to an interior wall of the left side half of the gun lock or welded thereto or the other half depending upon the orientation of the device. The corresponding servo shaft made up of portions 1420, 1430 and 1440 operates in circular cylindrical guide protrusions (similar to the guide mount protrusions found to hold item 1340 in FIG. 16B or the guides forming one continuous piece having holes) that have holes therein for the guiding of the servo shaft beginning and end portions 1420 and 1440. These holes serve to ensure the proper rotation of the servo shaft within the holes. The protrusions themselves are integral, welded or bolted to the interior surface of the gun lock and in a manner most befitting the particular implementation. Further the mount or protrusions themselves have openings in one side to permit the contact with the grooves of the main shaft 1410.

The servo shaft has a corresponding integrated toothed 1430 that is in contact with the continuous groove of the first shaft 1410. When the motor 1400 operates it turns its integral shaft 1410 that makes contact at its continuous groove with the teeth 1430 of the servo shaft. In so doing the motor can be controlled to extend or retract the shaft tip 1440 into the shaft teeth 1330 of FIG. 13 upon detection of an appropriate close (FIG. 14b) or open signal (FIG. 14a) as discussed above in Keogh. In either case, an end stopper 1460 operates to stop the complete forward motion of the shaft in cooperation with a disk 1450 that is integral with the servo shaft at shown so that the part does not disengage. The stop 1460 is bolted to, welded or otherwise made integrally from the interior walls of the gun lock. Of course, the operation of the circuit presumes that the motor 1400 of FIGS. 14a and 14b are connected conventionally as in the Keogh circuit and software routines described previously.

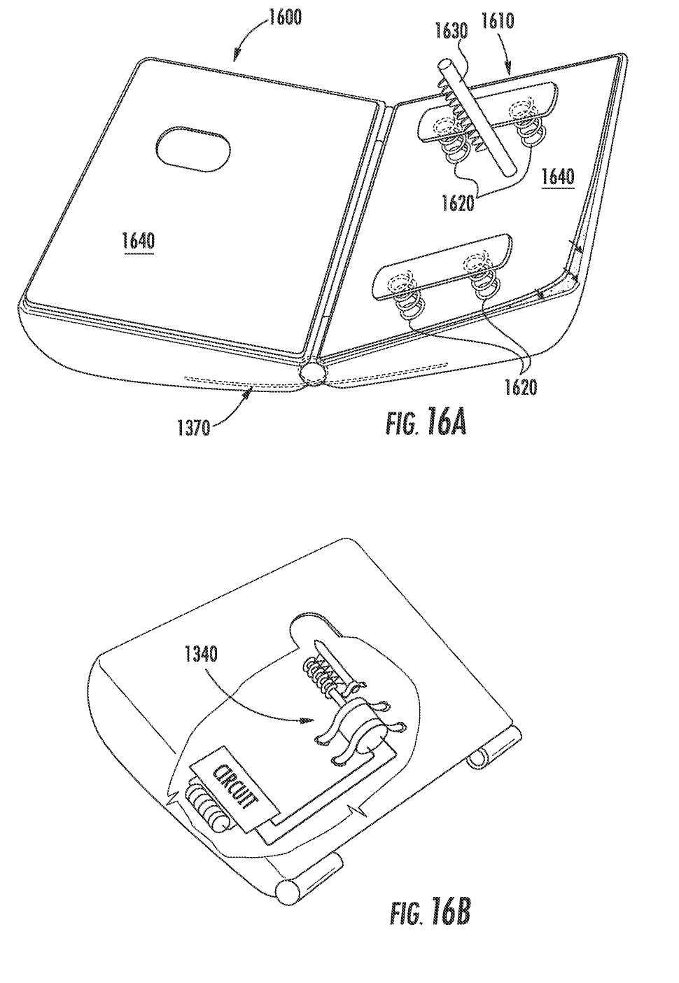

FIG. 15 illustrates a solenoid utilized in an optional version of the embodiment that are internal to the Biometric Quick Release Gun Lock. A solenoid coil 1530 is connected across the electric circuits discussed previously in Keogh and acts as a convenient optional mechanism for engaging the teeth of the external shaft 1330 that extends from one side 1310 of the gun lock to the other 1300. The solenoid is bolted, welded or weld mounted to a convenient inner surface as would the motor of FIG. 14. In a typical solenoid a base 1500 is permanently connected to the interior of the solenoid so as to prevent its motion. A spring 1510 with an appropriate spring constant that is welded or permanently connected to the base 1500 extends the servo shaft 1520 in an electrically off condition as shown in FIG. 15A. When energized by the thumbprint recognition previously discussed the solenoid is activated retracting the servo shaft 1520 inwards thereby compressing the spring 1510 as shown in FIG. 15B. As a result the servo shaft tip no longer contacts the teeth of the shaft 1330 that extends externally from the exterior of the two halves of the gun lock. Further, an end stopper 1550 operates to stop the complete forward motion of the shaft in cooperation with a disk 1540 that is integral with the servo shaft at shown so that the part does not disengage. The stop 1550 is bolted to, welded or otherwise made integrally from the interior walls of the gun lock. Of course, the operation of the circuit presumes that the solenoid 1530 of FIGS. 15a and 15b are connected conventionally as in the circuit described previously in Keogh software and hardware.

FIG. 16a illustrates two halves of the gun lock 1600 and 1610 that have a pair of dual spring mounted (metal-aluminum or alloy steel) oblong pieces 1620 associated there between; this spring mounted pieces 1620 are welded or inserted into slots that tie down the spring at the ends using excess material from the spring 1620 ends themselves and turned using hand tools or welding guns. The oblong mounts are also similarly connected to the other end of the springs as shown and are disposed along the front and back borders of the lock. The (metallic, aluminum or steel alloy) springs are of an appropriate spring constant to quickly open the gun-lock should the biometric fingerprint software and hardware determine that an authorized user has been detected. Additionally a rubber seal 1640 is affixed to some or all of the exterior surfaces of the gun lock to prevent damage to the gun lock as it carried about or laid down on top of hard surfaces. The rubber seal is glued or affixed using adhesives; alternatively, pressure contact of parts that are compressed using simple mechanical pressure into convenient tabs that act similarly to the reverse side of a photo album whereby multiple tabs act to hold the seal in place or similar mechanisms may be utilized such as hollowed out ridges along the surface of the gun lock that receive the ends of the rubber seal along convenient zones. Item 1370 represents a spring (FIG. 13B, 1370, FIG. 16A, 1370—threaded internally between the two halves, wound about the hinge and across an opening in the hinge cylinder portions and positioned internally against the internal surface of the external faces on either side of the gun lock halves so as to assist the springing motion to open the two halves) that permits the rapid opening of the Biometric Quick Release Gun Lock 1200 when a properly authorized thumbprint has been presented to the concave thumbprint scanning biometric area 1250. The spring passes through holes in either half and in the portion of the hinge it is to be wound about or pass through depending upon the implementation to engage its springing action as needed.

FIG. 16b illustrates a cutaway showing the interior of the gun lock having electronics on a circuit card, motor, shaft and servo shaft that acts to close access to the gun lock as it extends parallel to the hole in the surface of the lock as shown. Options for the fixed of the different components to the interiors of either half include welding to integral mounts, bolting of the devices with screws, welding to mounts welded or bolted onto an inner surface of a half of the gun lock device or the use of mount guides like that shown in FIG. 16B item 1340's curved holders. The toothed shaft of FIG. 16A that is permanently connected to one side enters a hollow cavity as shown in FIG. 16A-B on the other side of the gun lock and makes contact with the servo shaft. This cavity is of a size that permits the easy entrance of the shaft. Of course the shaft and other parts are not to scale and the drawings are intended only for informational purposes as to the functioning of the device. Thus, an actual working shaft, cavity etcetera can be smaller or larger depending upon the particular fielded device.

FIG. 16C shows the positioning of protrusions and matching cavities; the tongue protrusions fit into matching cavities thereby ensuring that the device will not disengage from the trigger guard. For example, protrusion 1650 slides into a matching cavity 1670 and protrusion 1660 slides into a matching cavity 1680. When appropriately positioned about the trigger finger guard each of the protrusions 1650 and 1660 easily prevent the gun-lock from slipping out of the trigger guard. Additionally, the two protrusions can be integrated into one single one or made of more than two protrusion and cavity pairs. Item 1690 represents the hinge shown between the two gun-lock halves.

Both halves of the gun lock are partially (where there are filled in sections of the interior for extra gun lock strength) or completely hollow shells (only a surface skin and interior surface skin for the halves) for acceptance of whatever components are suitable to a given implementation. The electronics of Keogh are arranged internally in the instant invention in such a fashion as to facilitate the scanning of thumbprints and the performance of the secure locking of the device; similarly, the software is utilized to permit the user to easily register, sense and discriminate authorized users from unauthorized users and to act upon such information accordingly. In the primary embodiment shown, the toothed shaft goes in front of the trigger and not behind; optional variations are behind the trigger and outside of the rounded finger guard that sits about the trigger. A user grasps the Quick Biometric Gun Lock and threads the shaft on one enclosed half shell through the opening in the gun's circular trigger guard and on into the cavity in the other enclosed half shell shown in FIG. 16A-B. The toothed shaft on one half shell automatically engages onto the servo shaft whether of a solenoid or motor helical groove varieties. The tongue protrusions 1650, 1660 help the proper positioning of the device in the circular trigger guard. Once the lock is clamped on the lock cannot slide back into trigger because it will be against the gun handle. The importance is that if a woman grabs the gun by the handle in her purse with her thumb on the reader by the time she lifts it to shoot the lock has already sprung off. This is the basic concept whether you take out of car glove box and are able to fire by the time the gun is raised to an attacker. There is a bumper like material on the inside of both sides that is semi hard rubber which squishes just a little when clamped on and doesn't allow for much movement. The invention has thus been described in such clear and precise terms as to enable one of ordinary skill in the art to understand its fundamental principles.

I claim:

1. A quick biometric gun lock comprising:
a hinge located between two sides such that the
two sides open and close about the hinge and
a cavity between the two sides for reception of the finger protector and trigger mechanism of a gun wherein the quick biometric gun lock encloses the trigger and finger protection area.

2. The quick biometric gun lock of claim 1, further comprising:
a control shaft attached to one of the sides having teeth for closure of the gun lock.

3. The quick biometric gun lock of claim 2, further comprising:
a control device in the other side for engaging the teeth of the control shaft.

4. The quick biometric gun lock of claim 3 wherein the control device is an electric motor and integral shaft moving a toothed servo shaft that is in contact with corresponding grooves of the electric motor.

5. The quick biometric gun lock of claim 4, further comprising:
a stopping disk integral with the servo shaft and
a stop integral with the interior of the gun lock.

6. The quick biometric gun lock claim 3 wherein the control device is a solenoid and integral shaft.

7. The quick biometric gun lock of claim 6, further comprising:
a stopping disk integral with the integral shaft and
a stop integral with the interior of the gun lock.

8. The quick biometric gun lock of claim 1, further comprising:
a portion extending from one side of the gun lock having a biometric fingerprint sensor.

9. The quick biometric gun lock of claim 1, further comprising:
a low battery detection speaker.

10. The quick biometric gun lock of claim 1, further comprising:
a low battery detection light.

11. The quick biometric gun lock of claim 1, further comprising:
a USB port utilized for both data transceiver operations and for recharging facilities.

12. The quick biometric gun lock of claim 1, further comprising:
a conventional power supply recharging port.

13. The quick biometric gun lock of claim 1, further comprising:
a rubber seal affixed to the gun lock's external surface.

14. The quick biometric gun lock of claim 1, further comprising:
a spring mounted bumper connected to one of the sides and operating against the two sides of the gun lock so as to open the gun lock.

15. The quick biometric gun lock of claim 1, further comprising:
a spring mounted internally to the two sides and configured to operate against the internal surface of the exterior face of the sides.

16. The quick biometric gun lock of claim 1, further comprising:
a protrusion integral with an interior external surface of one side and
a matching cavity on the other side for reception of the protrusion.

17. A quick biometric gun lock device comprising:
two half portions forming a main body of the gun lock connected by a hinge and integral with
a portion extending from one side of the gun lock having a biometric fingerprint sensor.

18. The quick biometric gun lock device of claim 17, further comprising:
an oblong spring mounted bumper connected to one half portion.

19. The quick biometric gun lock device of claim 17, further comprising:
an internal spring connected to the hinge and in contact with both half portions.

20. A quick biometric gun lock device comprising:
two half portions forming a main body connected by
a hinge and
a device for holding the two portions together in cooperation with
a toothed shaft integral with one portion and extending from one portion to another.
